(19) United States
(12) Patent Application Publication
(10) Pub. No.: US 2011/0118229 A1
(43) Pub. Date: May 19, 2011

(54) 2-ARYLMETHYLAZETIDINE-CARBAPENEM-3-CARBOXYLIC ACID ESTER DERIVATIVE OR ITS SALT, PROCESS FOR THE PREPARATION THEREOF AND PHARMACEUTICAL COMPOSITION COMPRISING THE SAME

(75) Inventors: Young-Ro Choi, Seoul (KR); Bong-Jin Kim, Daejeon (KR); Bok-Ju Song, Daejeon (KR); Bum-Soo Lee, Seoul (KR); Dong-Woo Lee, Seoul (KR); Dong-Geun Seo, Incheon (KR); Young-Cheol Jeong, Gyeonggi-do (KR); Si-Min Kim, Gyeonggi-do (KR); Jee-Woong Kwon, Daejeon (KR); Jae-Yang Kong, Seoul (KR); Heeyeong Cho, Daejeon (KR)

(73) Assignees: KUKJE PHARM. IND. CO., LTD., Seongnam-si, Gyeonggi-do (KR); KOREA RESEARCH INSTITUTE OF CHEMICAL TECHNOLOGY, Daejeon (KR)

(21) Appl. No.: 12/742,966

(22) PCT Filed: Nov. 18, 2008
(86) PCT No.: PCT/KR08/06782

§ 371 Date: May 14, 2010

(30) Foreign Application Priority Data
Nov. 23, 2007 (KR) 10-2007-0120038
Mar. 28, 2008 (KR) 10-2008-0028691

(51) Int. Cl. A61K 31/407; C07D 477/20; C07D 477/08; C07D 477/06; A61P 31/04

(52) U.S. Cl. 514/210.12; 540/350

(57) ABSTRACT

The present invention provides a 2-arylmethylazetidine-carbapenem-3-carboxylic acid ester derivative or its pharmaceutically acceptable salt, a process for the preparation thereof, and a pharmaceutical composition comprising the same. The 2-arylmethylazetidine-carbapenem-3-carboxylic acid ester derivatives or their pharmaceutically acceptable salts show high oral absorption rate, and thus can be orally administered. The active metabolites thereof have a broad spectrum of antibacterial activities against Gram-positive and Gram-negative bacteria and excellent antibacterial activities against methicillin-resistant Staphylococcus aurus (MRSA) and quinolone-resistant strains (QRS). In particular, the acid addition salts of the 2-arylmethylazetidine-carbapenem-3-carboxylic acid ester derivatives are obtained in crystalline forms having excellent stability.

### TECHNICAL FIELD

The present invention relates to a 2-arylmethylazetidine-carbapenem-3-carboxylic acid ester derivative or its pharmaceutically acceptable salt, a process for the preparation thereof, and a pharmaceutical composition comprising the same.

### BACKGROUND ART

Among beta-lactam antibiotics, carbapenem antibiotics show very strong antibacterial activity and have excellent safety and therapeutic effect, thereby being used for children, feeble elderly people with immune function decreased, and patients suffering from serious illnesses. Furthermore, carbapenem antibiotics also show excellent antibacterial activity against resistant bacteria which are not easily cured, and thus used as medication therefor.

Imipenem and meropenem, which are being marketed as a carbapenem antibiotic with a broad spectrum of antibacterial activities, are usually administered to patients suffering from serious illnesses. However, imipenem and meropenem are only parenterally used. Even though many researchers have attempted to develop an orally administrable carbapenem compound for improving patients' compliance, there has not been yet marketed an orally administrable carbapenem compound. For oral use, tebipenem derivatives, in the form of carbapenem ester prodrug, are being developed (see U.S. Pat. No. 5,783,703) and clinical trial (phase III) thereof are being conducted.

The present inventors have disclosed 2-arylmethylazetidine-carbapenem-3-carboxylic acid of the following formula having a broad spectrum of antibacterial activities against Gram-negative and Gram-positive bacteria; and excellent antibacterial activities against resistant bacteria, such as methicillin-resistant Staphylococcus aureus (MRSA) (WO2006/025634 and KR Patent No. 10-0599876).

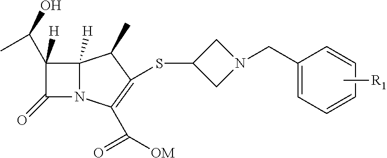

wherein R₁ is a hydrogen atom, a C₁-C₃ alkyl group, a C₁-C₃ alkyloxy group, a hydroxyl group, an amine group, an alkylamine group, an alkylthiol group, a trifluoromethyl group, or a halogen atom; M is a hydrogen atom or an alkali metal group.

The compounds, obtained by introducing arylmethylazetidine group at the 2-position of the carbapenem skeleton, show a broad spectrum of antibacterial activities and have antibacterial activities against resistant strains. Furthermore, the compounds are stable to renal dehydropeptidase-1; show excellent pharmacokinetic properties; and have a favorable safety profile in toxicity studies, e.g., nephrotoxicity study.

### DETAILED DESCRIPTION OF THE INVENTION

### Technical Problem

The present invention provides a carbapenem derivative or its pharmaceutically acceptable salt, particularly an acid addition salt, which is orally administrable and has high chemical stability. The carbapenem derivative or its pharmaceutically acceptable salt also shows a broad spectrum of antibacterial activities and excellent antibacterial activities against resistant strains.

The present invention also provides a process for preparing the carbapenem derivative or its pharmaceutically acceptable salt.

The present invention also provides an antibiotic composition comprising the carbapenem derivative or its pharmaceutically acceptable salt as an active ingredient.

### Technical Solution

The present invention provides an ester derivative of 2-arylmethylazetidine-carbapenem-3-carboxylic acid, or its pharmaceutically acceptable salt, which is obtained by introducing a compound having a particular structure at 3-position of 2-arylmethylazetidine-carbapenem-3-carboxylic acid via an ester bond; a process for the preparation thereof; and a pharmaceutical composition including the same. The 2-arylmethylazetidine-carbapenem-3-carboxylic acid ester derivatives or their pharmaceutically acceptable salts show high oral absorption rate and thus can be orally administered. The active metabolite thereof has a broad spectrum of antibacterial activities against Gram-positive and Gram-negative bacteria and excellent antibacterial activities against methicillin-resistant Staphylococcus aureus (MRSA) and quinolone-resistant strains (QRS). In particular, the acid addition salts of the 2-arylmethylazetidine-carbapenem-3-carboxylic acid ester derivatives are obtained in crystalline forms having high chemical stability.

According to an aspect of the present invention, there is provided a carbapenem derivative of Formula 1 or its pharmaceutically acceptable salt:

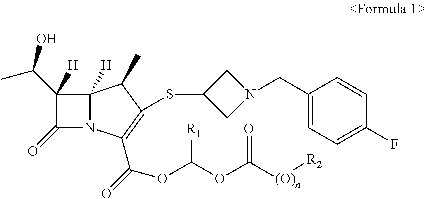

wherein, R₁ is a hydrogen atom or a C₁-C₄ alkyl group; R₂ is a linear or branched C₁-C₁₂ alkyl group optionally substituted with C₄-C₇ cycloalkyl, or a C₄-C₇ cycloalkyl group optionally substituted with C₁-C₄ alkyl; and n is 0 or 1. Preferably, the pharmaceutically acceptable salt is an acid addition salt of the carbepenem derivative of Formula 1.

According to another aspect of the present invention, there is provided a process for preparing a carbapenem derivative of Formula 1 or its pharmaceutically acceptable salt, which comprises reacting a compound of Formula 2 with a compound of Formula 3:

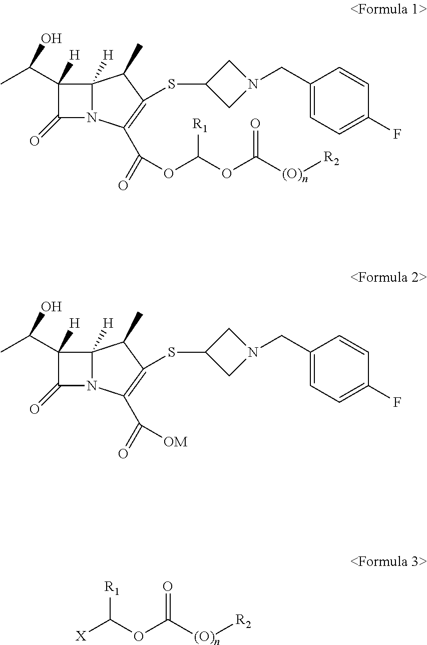

wherein, M is a hydrogen atom or an alkali metal; X is a halogen atom; and R₁, R₂, and n is the same as defined in the above.

According to still another aspect of the present invention, there is provided a process for preparing an acid addition salt of a carbapenem derivative of Formula 1, which comprises reacting a carbapenem derivative of Formula 1 with an acid:

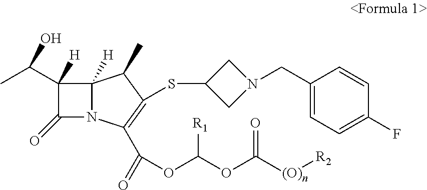

wherein, R₁, R₂, and n is the same as defined in the above.

According to still another aspect of the present invention, there is provided an antibiotic composition comprising the carbapenem derivative of Formula 1 or its pharmaceutically acceptable salt as an active ingredient; and a pharmaceutically acceptable carrier.

### Advantageous Effects

The 2-arylmethylazetidine-carbapenem-3-carboxylic acid ester derivatives or their pharmaceutically acceptable salts according to the present invention show high oral absorption rate, and thus can be orally administered. The active metabolite thereof has a broad spectrum of antibacterial activities against Gram-positive and Gram-negative bacteria and excellent antibacterial activities against methicillin-resistant Staphylococcus aurus (MRSA) and quinolone-resistant strains (QRS). In particular, the acid addition salts of the 2-arylmethylazetidine-carbapenem-3-carboxylic acid ester derivatives are obtained in crystalline forms having high chemical stability. The acid addition salts in crystalline forms may be stored for a long period of time due to their high stability. And also, those show oral absorption about 2.7 times higher than that of their free base forms.

### DESCRIPTION OF THE DRAWINGS

FIG. 1 illustrates the results of acute toxicity test of the compound prepared in Example 10.

FIG. 2 illustrates the results of acute toxicity test of the compound prepared in Example 17.

### BEST MODE FOR CARRYING OUT THE INVENTION

The present invention includes a carbapenem derivative of Formula 1 or its pharmaceutically acceptable salt:

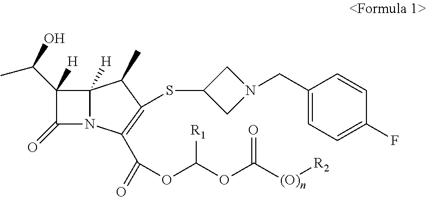

wherein, R₁ is a hydrogen atom or a C₁-C₄ alkyl group; R₂ is a linear or branched C₁-C₁₂ alkyl group optionally substituted with C₄-C₇ cycloalkyl, or a C₄-C₇ cycloalkyl group optionally substituted with C₁-C₄ alkyl; and n is 0 or 1.

In the carbepenem derivative of Formula 1 or its pharmaceutically acceptable salt, preferably R₁ is a hydrogen atom or a C₁-C₄ alkyl group; R₂ is a linear or branched C₁-C₁₀ alkyl group, a C₄-C₇ cycloalkylmethyl group, a C₄-C₇ cycloalkyl group, or a C₄-C₇ cycloalkyl group substituted with a C₁-C₄ alkyl group; and n is 0 or 1. More preferably, to R₁ is a hydrogen atom or a methyl group; R₂ is a methyl group, a t-butyl group, an isobutyl group, an isopropyl group, an n-hexyl group, an n-nonyl group, a cyclohexylmethyl group, a cyclohexyl group, or a 1-methylcyclohexyl group; and n is 0 or 1.

It is preferable that the pharmaceutically acceptable salt is an acid addition salt of the carbepenem derivative of Formula 1. The acid addition salt may be an addition salt of the inorganic acid selected from the group consisting of hydrochloric acid, phosphoric acid, sulfuric acid, hydrobromic acid, hydroiodic acid, and nitric acid; or an addition salt of the organic acid selected from the group consisting of acetic acid, propionic acid, butyric acid, trifluoroacetic acid, trichloroacetic acid, fumaric acid, maleic acid, lactic acid, methanesulfonic acid, trifluoromethanesulfonic acid, benzoic acid, p-nitrobenzoic acid, benzenesulfonic acid, p-nitrobenzenesulfonic acid, p-bromobenzenesulfonic acid, toluenesulfonic acid, 2,4,6-triisopropylbenzenesulfonic acid, and diphenyiphosphinic acid. Preferably, the acid addition salt is an addition salt of phosphoric acid, hydrochloric acid, maleic acid, fumaric acid, benzenesulfonic acid, p-toluenesulfonic acid, trifluoroacetic acid, or lactic acid.

Examples of the carbepenem derivatives of Formula 1 or their pharmaceutically acceptable salts are:

pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;

1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;

1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;

cyclohexylacetoxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;

(1-methylcyclohexanecarboxy)methyl(1R,5S,6S)-2-[(1-(4-fluorobenzyl)azetidin-3-yl)thio]-6-[(R)-1-hydroxyethyl]-1-methyl-carbapen-2-em-3-carboxylate;

isovaleroylmethyl(1R,5S,6S)-2-[(1-(4-fluorobenzyl)azetidin-3-yl)thio]-6-[(R)-1-hydroxyethyl]-1-methyl-carbapen-2-em-3-carboxylate;

n-decanoyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;

1-(n-hexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;

1-(acetoxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;

phosphoric acid salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;

hydrochloric acid salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;

hydrochloric acid salt of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;

hydrochloric acid salt of 1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;

maleic acid salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;

maleic acid salt of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;

maleic acid salt of 1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;

fumaric acid salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;

fumaric acid salt of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;

fumaric acid salt of 1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;

benzenesulfonic acid salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;

benzenesulfonic acid salt of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;

benzenesulfonic acid salt of 1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;

p-toluenesulfonic acid salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;

p-toluenesulfonic acid salt of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;

p-toluenesulfonic acid salt of 1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;

trifluoroacetic acid salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;

trifluoroacetic acid salt of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;

trifluoroacetic acid salt of 1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;

lactic acid salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;

lactic acid salt of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;

lactic acid salt of 1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate.

Examples of preferable carbepenem derivatives of Formula 1 or their pharmaceutically acceptable salts are:

pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate or its acid addition salt;

1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate or its acid addition salt;

1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate or its acid addition salt.

More preferable compound in the carbepenem derivative of Formula 1 or its pharmaceutically acceptable salts is a phosphoric acid salt of pivaloyloxymethyl (1R,5S,6S)-2-[(1-(4-fluorobenzyl)azetidin-3-yl)thio]-6-[(R)-1-hydroxyethyl]-1-methyl-carbapen-2-em-3-carboxylate.

When the carbepenem derivative of Formula 1 or its pharmaceutically acceptable salt according to the present invention is orally administered, it is absorbed through the gastrointestinal tract in high absorption rate and then metabolized into 2-arylmethylazetidine-carbapenem-3-carboxylic acid, that is (1R,5S,6S)-2-[(1-(4-fluorobenzyl)azetidin-3-yl)thio]-6-[(R)-1-hydroxyethyl]-1-methyl-carbapen-2-em-3-carboxylic acid. The (1R,5S,6S)-2-[(1-(4-fluorobenzyl)azetidin-3-yl)thio]-6-[(R)-1-hydroxyethyl]-1-methyl-carbapen-2-em-3-carboxylic acid has a broad spectrum of antibacterial activities against Gram-positive and Gram-negative bacteria and has excellent antibacterial activities against methicillin-resistant Staphylococcus aureus (MRSA) and quinolone-resistant strains (QRS). In particular, the acid addition salt of the carbepenem derivative of Formula 1 is obtained in a crystalline form, and the crystalline form has excellent stability.

The present invention also provides a process for preparing the carbepenem derivative of Formula 1 or its pharmaceutically acceptable salt. That is, the present invention provides a process for preparing a carbapenem derivative of Formula 1 or its pharmaceutically acceptable salt, which comprises reacting a compound of Formula 2 with a compound of Formula 3:

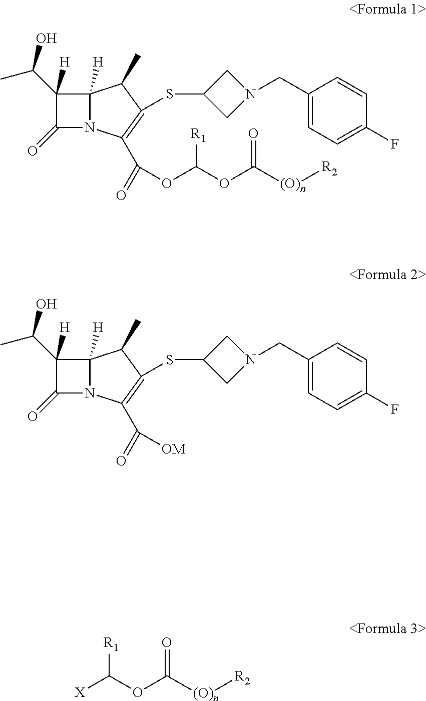

wherein, M is a hydrogen atom or an alkali metal; X is a halogen atom; and R₁, R₂, and n is the same as defined in the above.

The carbapenem derivative of Formula 1 or its pharmaceutically acceptable salt is prepared according to Reaction Scheme 1 below.

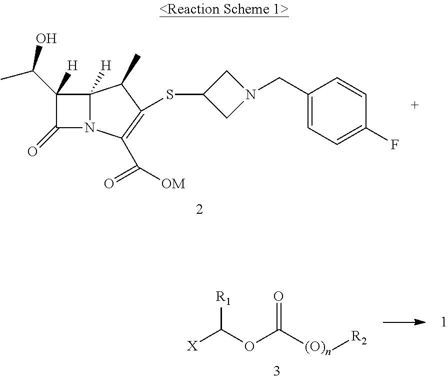

In Reaction Scheme 1, M is a hydrogen atom or an alkali metal (preferably sodium or potassium, more preferably potassium); X is a halogen atom (preferably chloro or iodo); and R₁, R₂ and n are the same as defined in the above.

The compound of Formula 2 may be prepared in the same manner as in WO2006/025634 (and KR Patent No. 10-0599876). If necessary, the compound of Formula 2 of an alkali metal salt form may be converted into its free base form by regulating pH of an aqueous solution thereof. The compound of Formula 3 may be prepared in the same manner as in U.S. Pat. No. 5,886,172.

The reaction between the compounds of Formulae 2 and 3 may be conducted in the presence of a base. The base includes at least one selected from the group consisting of: an inorganic base such as sodium hydroxide, potassium hydroxide, sodium carbonate, potassium carbonate, sodium bicarbonate, and potassium bicarbonate; and an organic base such as triethylamine, N,N-diisopropylethylamine, and pyridine. For example, the base may be triethylamine and/or potassium carbonate.

And also, the reaction between compounds of Formulae 2 and 3 may be conducted in the presence of a quaternary ammonium salt, in addition to the base. The quaternary ammonium salt includes tetraethylammonium chloride, tetrabutylammonium chloride, tetrabutylammonium bromide, benzyltriethylammonium chloride, or the like.

In addition, the reaction between compounds of Formulae 2 and 3 may be conducted in a solvent, e.g. an ether such as diethyl ether, tetrahydrofuran, and dioxane; a hydrocarbon such as toluene, xylene, and cyclohexane; a halogenated hydrocarbon such as dichloromethane and chloroform; N,N-dimethylformamide; N,N-dimethylacetamide; acetonitrile; or dimethylsulfoxide. The solvent may be N,N-dimethylformamide and/or N,N-dimethylacetamide.

The compound of Formula 3 may be used in a range of 1 to 3 mole equivalents, preferably 1 to 2 mole equivalents, based on 1 mole equivalent of the compound of Formula 2, but is not limited thereto. In addition, the base and the quaternary ammonium salt may respectively be used in a range of 1 to 3 mole equivalents, based on 1 mole equivalent of the compound of Formula 2, but are not limited thereto.

In the reaction between the compounds of Formulae 2 and 3, the temperature may be in a range of about −20° C. to about 75° C., but is not limited thereto. For example, if the substituent X of the compound of Formula 3 is chlorine, the reaction may be conducted in a temperature ranging from 40° C. to 75° C. If the substituent X is iodine, the reaction may be conducted in a temperature ranging from −20° C. to 40° C. The reaction may be conducted for 10 minutes to 2 hours, but the reaction time is not limited thereto.

The compound of Formula 1 prepared according to the process of the present invention may be isolated and purified using a conventional isolation and purification method. For example, the compound of Formula 1 may be isolated from a reaction mixture; and then purified according to a conventional method, e.g., extraction, washing, concentration under a reduced pressure, column chromatography, recrystallization, or the like.

According to an embodiment of the present invention, there is provided a process for preparing an acid addition salt of the carbepenem derivative of Formula 1. That is, the present invention provides a process for preparing an acid addition salt of a carbapenem derivative of Formula 1, which comprises reacting a carbapenem derivative of Formula 1 with an acid:

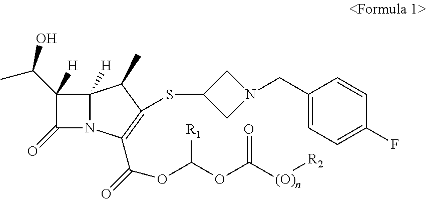

wherein, R₁, R₂, and n is the same as defined in the above. The carbepenem derivative of Formula 1 used as a starting material for the process for preparing the acid addition salt of the carbapenem derivative may be prepared in the same manner as the process described above.

The acid may be an inorganic acid selected from the group consisting of hydrochloric acid, phosphoric acid, sulfuric acid, hydrobromic acid, hydroiodic acid, and nitric acid; or an organic acid selected from the group consisting of acetic acid, propionic acid, butyric acid, trifluoroacetic acid, trichloroacetic acid, fumaric acid, maleic acid, lactic acid, methanesulfonic acid, trifluoromethanesulfonic acid, benzoic acid, p-nitrobenzoic acid, benzenesulfonic acid, p-nitrobenzenesulfonic acid, p-bromobenzenesulfonic acid, toluenesulfonic acid, 2,4,6-triisopropylbenzenesulfonic acid, and diphenylphosphinic acid. Preferably, the acid is phosphoric acid, hydrochloric acid, maleic acid, fumaric acid, benzenesulfonic acid, p-toluenesulfonic acid, trifluoroacetic acid, or lactic acid. More preferably, the acid is phosphoric acid.

The formation of the acid addition salt may be conducted in at least one organic solvent selected from the group consisting of acetone, ethyl acetate, isopropyl alcohol, tetrahydrofuran, and acetonitrile. For example, the acid addition salt may be formed by dissolving the carbapenem derivative of Formula 1 in the organic solvent, and adding an inorganic acid or an organic acid to the solution. The acid addition salt may be crystallized using water, n-hexane, methylene chloride/n-hexane, or ethyl acetate/n-hexane.

The acid may be used in a range of 1 to 3 mole equivalents, preferably 1 to 2 mole equivalents, based on 1 mole equivalent of the carbepenem derivative of Formula 1, but is not limited thereto. In addition, the temperature of the reaction between the carbepenem derivative of Formula 1 and the acid may be in a range of about −20° C. to about 50° C., but is not limited thereto. For example, when the acid is an inorganic acid, the reaction may be performed in a temperature ranging from 0° C. to 30° C. When the acid is an organic acid, the reaction may be performed in a temperature ranging from −20° C. to 50° C. The reaction time may be from 10 minutes to 5 hours, but is not limited thereto.

The acid addition salt of the carbepenem derivative of Formula 1 prepared according to the above process may be isolated and purified using a conventional isolation and purification method. For example, the acid addition salt of the compound of Formula 1 may be isolated from a reaction mixture; and then purified according to a conventional method, e.g., extraction, washing, concentration under a reduced pressure, recrystallization, or the like.

The acid addition salt of the carbepenem derivative of Formula 1 is obtained in a crystalline powder form, and the crystalline powder form has high chemical stability.

The present invention also provides an antibiotic composition comprising the carbapenem derivative of Formula 1 or its pharmaceutically acceptable salt as an active ingredient; and a pharmaceutically acceptable carrier. Preferably, the pharmaceutically acceptable salt of the carbapenem derivative is an acid addition salt of the carbepenem derivative of Formula 1. More preferably, the pharmaceutically acceptable salt of the carbapenem derivative is a phosphoric acid salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate.

The antibiotic composition may include 0.1 to 75% by weight, preferably 1 to 50% by weight, of the carbapenem derivative of Formula 1 or its pharmaceutically acceptable salt, based on the total weight of the pharmaceutical composition.

The antibiotic composition may be orally or parenterally administered, preferably orally administered. An oral formulation may be in the form of a tablet, pill, soft or hard capsule, solution, suspension, emulsion, syrup, powder, granule, or the like, and the formulation may include diluents (e.g.: lactose, dextrose, sucrose, mannitol, sorbitol, cellulose, and glycine), and lubricants (e.g.: silica, talc, stearic acid or a magnesium or calcium salt thereof, and polyethylene glycol). The tablet may include binders such as magnesium aluminum silicate, starch paste, gelatin, tragacanth, methylcellulose, sodium carboxy methylcellulose and polyvinyl pyrrolidine. The formulation may further include disintegrants such as starch, agar, alginic acid or a sodium salt thereof, and/or absorbents, colorants, flavoring agents, and sweeteners. The composition may be formulated by using a conventional method such as mixing, granulating and coating methods.

In addition, the pharmaceutical composition may be an injection formulation, preferably an isotonic solutions or suspension. The pharmaceutical composition may be sterilized and/or include additives such as preservatives, stabilizers, wetting agents, emulsifiers, salts or buffers for osmotic control and any other therapeutically useful materials.

A typical daily dose of the carbepenem derivative of. Formula 1 or its pharmaceutically acceptable salt may range from 2.5 to 200 mg/kg (body weight), preferably 5 to 100 mg/kg (body weight) in case of mammals including human, and may be administered in a single dose or in divided doses orally or parenterally.

The following examples are intended to further illustrate the present invention without limiting its scope of the present invention.

### PREPARATION EXAMPLE 1

### Preparation of 1-iodoethyl isopropylcarbonate

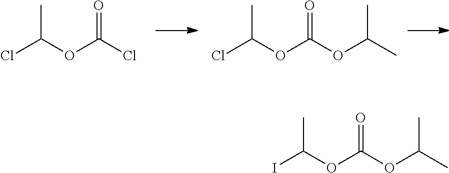

### (1) Preparation of 1-chloroethyl isopropylcarbonate

1-Chloroethyl chloroformate (31.7 g, 0.22 mol) was dissolved in methylene chloride (200 ml), and isopropanol (39.7 ml, 0.52 mol) was added thereto while ice-cooling. Pyridine (23 ml, 0.28 mol) was slowly added to the reaction mixture over 15 minutes. The reaction mixture was slowly heated to room temperature and then stirred for 30 minutes. The reaction mixture was sequentially washed with water, 5% brine, and 5% potassium hydrogen sulfate solution, dried over anhydrous magnesium sulfate, and then filtered. The filtrate was distilled under a reduced pressure to obtain 25 g of 1-chloroethyl isopropylcarbonate (yield: 68%).

bp_{55 mmHg}: 92-94° C.;

¹H-NMR (200 MHz, CDCl₃) δ 1.33 (d, J=6.0 Hz, 6H), 1.79 (d, J=6.0 Hz, 3H), 4.84 (heptet, J=6.0 Hz, 1H), 6.37 (q, J=6.0 Hz, 1H)

### (2) Preparation of 1-iodoethyl isopropylcarbonate

1-Chloroethyl isopropylcarbonate (13 g, 78 mmol) prepared in Step (1) was dissolved in acetonitrile (40 ml), and sodium iodide (4.2 g, 280 mmol, 3.58 eq) was added thereto. The reaction mixture was stirred at 60° C. for 70 minutes and then cooled to room temperature. The reaction mixture was distilled under a reduced pressure to remove the solvent. The resulting residue was extracted with water and ethyl acetate. The separated organic layer was washed with 5% sodium thiosulfate solution, dried over anhydrous magnesium, and then filtered. The filtrate was distilled under a reduced pressure to obtain 12.3 g of 1-iodoethyl isopropylcarbonate. The product was immediately used in subsequent reactions due to its instability.

¹H-NMR (200 MHz, CDCl₃) δ 1.33 (d, J=6.0 Hz, 6H), 2.28 (d, J=6.0 Hz, 3H), 4.82 (heptet, J=6.0 Hz, 1H), 6.43 (q, J=6.0 Hz, 1H)

### PREPARATION EXAMPLE 2

### Preparation of 1-iodoethyl cyclohexylcarbonate

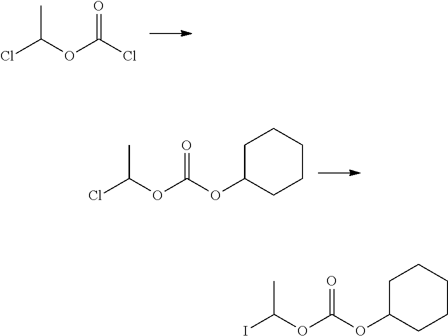

### (1) Preparation of 1-chloroethyl cyclohexylcarbonate

Cyclohexanol (19 ml, 0.18 mol) was dissolved in methylene chloride (300 ml), and pyridine (14.8 ml, 0.18 mol) was added thereto while ice-cooling. 1-Chloroethyl chloroformate (20 ml, 0.185 mol) was slowly added to the reaction mixture over 15 minutes. The reaction mixture was slowly heated to room temperature and then stirred for 16 hours. The reaction mixture was sequentially washed with water, brine, and 5% sodium thiosulfate solution, dried over anhydrous magnesium, and then filtered. The filtrate was distilled under a reduced pressure to obtain 26.06 g of 1-chloroethyl cyclohexylcarbonate (yield: 70%).

bp_{55 mmHg}: 101-103° C.;

¹H-NMR (200 MHz, CDCl₃) δ 1.0-2.3 (m, 10H), 1.38 (d, J=5.8 Hz, 3H), 4.60-4.80 (m, 1H), 6.40 (q, J=5.8 Hz, 1H).

### (2) Preparation of 1-iodoethyl cyclohexylcarbonate

1-chloroethyl cyclohexylcarbonate (2.6 g, 13 mmol) prepared in Step (1) was dissolved in acetonitrile (80 ml), and sodium iodide (8.5 g, 56.7 mmol, 4.36 eq) was added thereto. The reaction mixture was stirred at 60° C. for 70 minutes and then filtered. The filtrate was cooled to room temperature and then distilled under a reduced pressure to remove the solvent. The resulting residue was extracted with water and diethyl ether. The separated organic layer was washed with 5% sodium thiosulfate solution, dried over anhydrous magnesium sulfate, and then filtered. The filtrate was distilled under a reduced pressure to obtain 2.68 g of 1-iodoethyl cyclohexylcarbonate. The product was immediately used in subsequent reactions due to its instability.

¹H-NMR (200 MHz, CDCl₃) δ 0.9-2.2 (m, 10H), 2.20 (d, J=5.8 Hz, 3H), 4.60-4.80 (m, 1H), 6.81 (q, J=5.8 Hz, 1H).

### PREPARATION EXAMPLE 3

### Preparation of Iodomethyl Pivalate

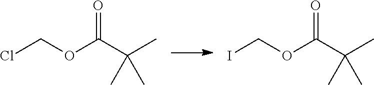

Chloromethyl pivalate (15.0 g, 0.1 mol) and sodium iodide (65 g, 0.43 mol) were dissolved in acetonitrile (600 ml), and then 22.5 g of iodomethyl pivalate was prepared in the same manner as in Preparation Example 2 (yield: 93%).

¹H-NMR (200 MHz, CDCl₃) δ 1.24 (s, 9H), 5.92 (s, 2H).

### PREPARATION EXAMPLE 4

### Preparation of (1R,5S,6S)-2-[(1-(4-fluorobenzyl)azetidin-3-yl)thio]-6-[(R)-1-hydroxyethyl]-1-methyl-carbapen-2-em-3-carboxylic acid

Potassium (1R,5S,6S)-2-[(1-(4-fluorobenzyl)azetidin-3-yl)thio]-6-[(R)-1-hydroxyethyl]-1-methyl-carbapen-2-em-3-carboxylate (20 g, 44.9 mmol) prepared according to WO2006/025634 (Korean Patent No. 10-0599876) was dissolved in water (60 ml), and the pH was controlled to pH 5.5 using acetic acid. The reaction mixture was subjected to C18 reverse phase column chromatography (eluent: water, 10% acetonitrile/water, and 20% acetonitrile/water) for isolation and purification. The fraction was lyophilized to obtain 17.3 g of the titled compound as a white solid (yield: 95%, HPLC purity: 99%).

¹H-NMR (300 MHz, D₂O) δ 1.17d, J=7.3 Hz, 3H), 1.31d, J=6.1 Hz, 3H), 3.20, 1H), 3.41 (m, 1H), 3.69 (m, 2H), 4.07 (s, 1H), 4.18 (m, 5H), 7.20 (m, 2H), 7.40 (m, 2H).

### EXAMPLE 1

### Preparation of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate

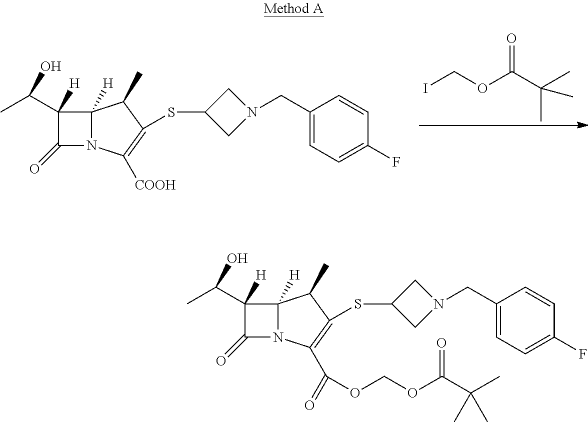

(1R,5S,6S)-2-[(1-(4-fluorobenzyl)azetidin-3-yl)thio]-6-[(R)-1-hydroxyethyl]-1-methyl-carbapen-2-em-3-carboxylic acid (1.77 g, 4 mmol) was dissolved in N,N-dimethylformamide (35 ml), and triethylamine (0.526 g, 5.2 mmol) and potassium carbonate powder (0.55 g, 4 mmol) were added thereto while ice-cooling. Iodomethyl pivalate (0.968 g, 4 mmol) prepared in Preparation Example 3 was slowly added to the reaction mixture, which was then stirred at the same temperature for 1 hour. The reaction mixture was further stirred for 1 hour while heating to room temperature. Water was added to the reaction mixture, which was then extracted with ethyl acetate. The separated organic layer was washed with brine, dried over anhydrous magnesium sulfate, and distilled under a reduced pressure to remove the solvent. The resulting residue was subjected to silica gel column chromatography (ethyl acetate:methanol=20:1, v/v) for purification to obtain 1.6 g of the title compound as a white powder (yield: 72%).

mp 65-67° C.;

¹H-NMR (200 MHz, CDCl₃) δ 1.18 (d, J=7.2 Hz, 3H), 1.23 (s, 9H), 1.32 (d, J=9.3 Hz, 3H), 3.01-3.17 (m, 1H), 3.18-3.28 (m, 3H), 3.59 (s, 2H), 3.64-3.80 (m, 2H), 3.82-4.01 (m, 2H), 4.12-4.28 (m, 2H), 5.90 (AB-q, 2H), 7.01 (m, 2H), 7.21 (m, 2H);

LCMS(m/e) 521(M⁺), 491, 407, 389, 320.

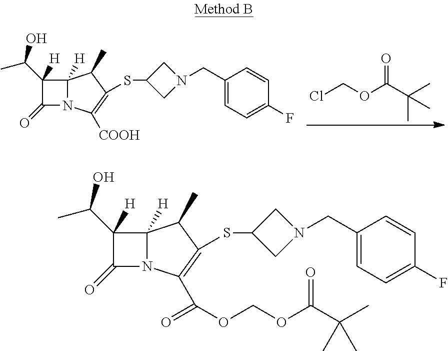

(1R,5S,6S)-2-[(1-(4-fluorobenzyl)azetidin-3-yl)thio]-6-[(R)-1-hydroxyethyl]-1-methyl-carbapen-2-em-3-carboxylic acid (2.0 g, 4.9 mmol), benzyltriethylammonium chloride (2.22 g, 9.8 mmol), and chloromethyl pivalate (1.47 g, 9.8 mmol) were dissolved in N,N-dimethylformamide (50 ml), and triethylamine (1.4 ml, 9.8 mmol) was added thereto. The reaction mixture was stirred at 65-70° C. for 2 hours. Water was added to the reaction mixture, which was then extracted with ethyl acetate. The separated organic layer was washed with brine, dried over anhydrous magnesium sulfate, and distilled under a reduced pressure to remove the solvent. The resulting residue was subjected to silica gel column chromatography (ethyl acetate:methanol=20:1, v/v) for purification to obtain 2.11 g of the title compound as a white powder (yield: 83%). As a result of analysis, the obtained product was the same as the product obtained according to Method A.

### EXAMPLE 2

### Preparation of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate

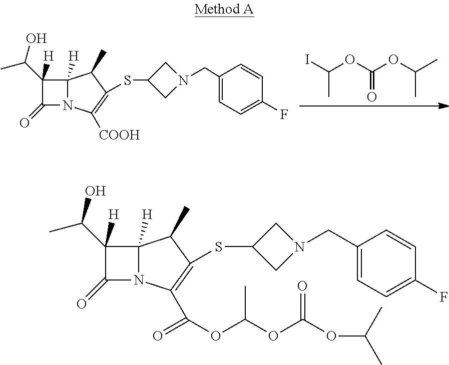

(1R,5S,6S)-2-[(1-(4-fluorobenzyl)azetidin-3-yl)thio]-6-[(R)-1-hydroxyethyl]-1-methyl-carbapen-2-em-3-carboxylic acid (1.0 g, 2.25 mmol) was dissolved in N,N-dimethylformamide (20 ml), and potassium carbonate powder (0.48 g, 4.5 mmol) was added thereto while ice-cooling. 1-Iodoethyl isopropylcarbonate (0.5 g, 2.47 mmol) prepared in Preparation Example 1 was slowly added to the reaction mixture, which was then stirred at the same temperature for 1 hour. The reaction mixture was further stirred for 1 hour while heating to room temperature. Water was added to the reaction mixture, which was then extracted with ethyl acetate. The separated organic layer was washed with brine, dried over anhydrous magnesium sulfate, and distilled under a reduced pressure to remove the solvent. The resulting residue was subjected to silica gel column chromatography (ethyl acetate:methanol=20:1, v/v) for purification to obtain 0.84 g of the title compound as a white powder (yield: 72%).

mp 85-87° C.;

¹H-NMR (200 MHz, CDCl₃) δ 1.18 (d, J=7.2 Hz, 3H), 1.31-1.61 (m, 9H), 1.33 (d, J=9.3 Hz, 3H), 1.48 (t, J=24 Hz, 3H), 2.05 (d, J=7.2 Hz, 3H), 3.02-3.08 (m, 1H), 3.18-3.28 (m, 2H), 3.59 (s, 2H), 3.64-3.80 (m, 2H), 3.82-4.01 (m, 2H), 4.12-4.28(m, 2H), 4.85-4.94 (m, 1H), 6.94 (m, 1H), 6.96-7.05 (m, 2H), 7.21-7.28 (m, 2H); LCMS(m/e) 521(M⁺), 496, 400, 389, 320.

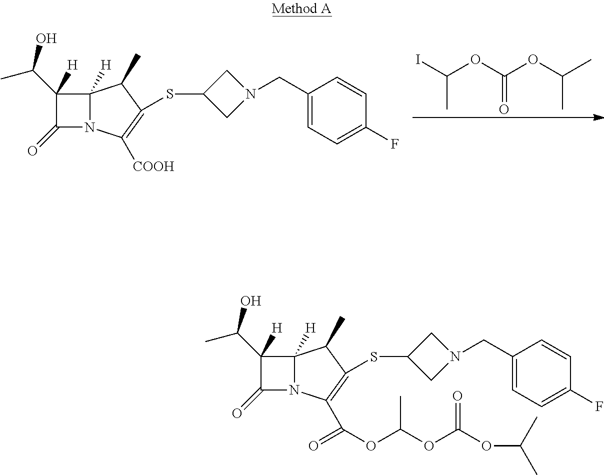

(1R,5S,6S)-2-[(1-(4-fluorobenzyl)azetidin-3-yl)thio]-6-[(R)-1-hydroxyethyl]-1-methyl-carbapen-2-em-3-carboxylic acid (2.0 g, 4.9 mmol), benzyltriethylammonium chloride (2.22 g, 9.8 mmol), and 1-iodoethyl isopropylcarbonate (1.6 g, 9.8 mmol) prepared in Preparation Example 1 were dissolved in N,N-dimethylformamide (50 ml), and triethylamine (1.4 ml, 9.8 mmol) was added thereto. The reaction mixture was stirred at 65-70° C. for 2 hours. The reaction mixture was cooled to room temperature. Water was added to the reaction mixture, which was then extracted with ethyl acetate. The separated organic layer was washed with brine, dried over anhydrous magnesium sulfate, and distilled under a reduced pressure to remove the solvent. The resulting residue was subjected to silica gel column chromatography (ethyl acetate:methanol=20:1, v/v) for purification to obtain 1.4 g of the title compound as a white powder (yield: 52%). As a result of analysis, the obtained product was the same as the product obtained according to Method A.

### EXAMPLE 3

### Preparation of 1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate

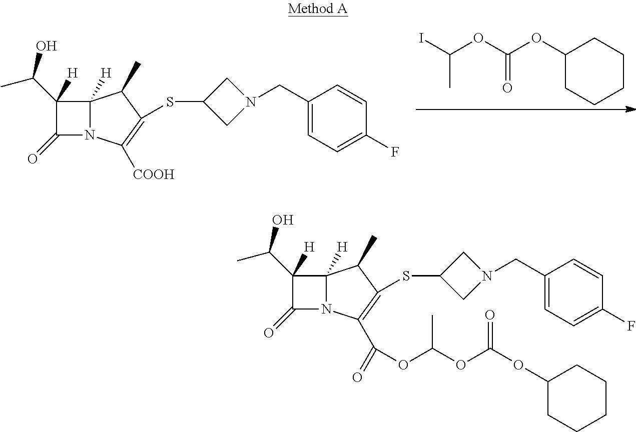

(1R,5S,6S)-2-[(1-(4-fluorobenzyl)azetidin-3-yl)thio]-6-[(R)-1-hydroxyethyl]-1-methyl-carbapen-2-em-3-carboxylic acid (1.0 g, 2.25 mmol) was dissolved in N,N-dimethylformamide (20 ml), potassium carbonate powder (0.48 g, 4.5 mmol) was added thereto while ice-cooling. 1-Iodoethyl cyclohexylcarbonate (0.74 g, 2.47 mmol) prepared in Preparation Example 2 was slowly added to the reaction mixture, which was then stirred at the same temperature for 1 hour. The reaction mixture was further stirred for 1 hour while heating to room temperature. Water was added to the reaction mixture, which was then extracted with ethyl acetate. The separated organic layer was washed with brine, dried over anhydrous magnesium sulfate, and distilled under a reduced pressure to remove the solvent. The resulting residue was subjected to silica gel column chromatography (ethyl acetate:methanol=20:1, v/v) for purification to obtain 1.06 g of the title compound as a white powder (yield: 82%).

mp 110-113° C.;

¹H-NMR (200 MHz, CDCl₃) δ 1.18 (d, J=7.2 Hz, 3H), 1.31-1.61 (m, 9H), 1.33-1.91 (m, 13H), 3.02-3.15 (m, 2H), 3.18-3.24 (m, 2H), 3.60 (s, 2H), 3.68-3.80 (m, 2H), 3.82-4.01 (m, 1H), 4.18-4.23 (m, 4H), 4.60-4.72 (m, 1H), 6.94 (m, 1H), 7.01 (t, 2H), 7.21-7.28 (m, 2H); LCMS(m/e) 577(M⁺), 428, 389, 320.

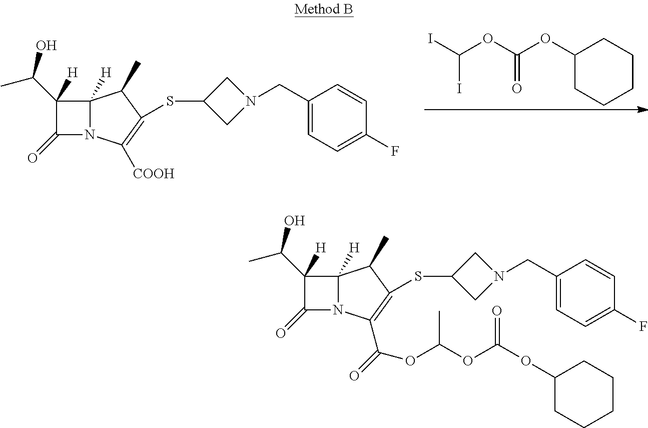

(1R,5S,6S)-2-[(1-(4-fluorobenzyl)azetidin-3-yl)thio]-6-[(R)-1-hydroxyethyl]-1-methyl-carbapen-2-em-3-carboxylic acid (2.0 g, 4.9 mmol), benzyltriethylammonium chloride (2.2 g, 9.8 mmol), and 1-chloroethyl cyclohexylcarbonate (2.0 g, 9.8 mmol) prepared in Preparation Example 2 were dissolved in N,N-dimethylformamide (50 ml), and triethylamine (1.4 ml, 9.8 mmol) was added thereto. The reaction mixture was stirred at 65-70° C. for 2 hours. The reaction mixture was cooled to room temperature. Water was added to the reaction mixture, which was then extracted with ethyl acetate. The separated organic layer was washed with 5% brine, dried over anhydrous magnesium sulfate, and distilled under a reduced pressure to remove the solvent. The resulting residue was subjected to silica gel column chromatography (ethyl acetate:methanol=20:1, v/v) for purification to obtain 1.9 g of the title compound as a white powder (yield: 70%). As a result of analysis, the obtained product was the same as the product obtained according to Method A.

Compounds of Examples 4 to 8 were prepared in the same manner as in Example 3, respectively using halogen-substitued derivatives of Formula 3 (see U.S. Pat. No. 5,886,172) of cyclohexylacetoxymethyl chloride, (1-methylcyclohexanecarboxy)methyl chloride, isovaleroylmethyl chloride, n-decanoyloxymethyl chloride, and 1-(n-hexyloxycarbonyloxy)ethyl chloride, as starting materials.

### EXAMPLE 4

### Preparation of cyclohexylacetoxymethyl(1R,5S,6S)-2-[(1-(4-fluorobenzyl)azetidin-3-yl)thio]-6-[(R)-1-hydroxyethyl]-1-methyl-carbapen-2-em-3-carboxylate

The title compound was prepared in the same manner as in Example 3, using (1R,5S,6S)-2-[(1-(4-fluorobenzyl)azetidin-3-yl)thio]-6-[(R)-1-hydroxyethyl]-1-methyl-carbapen-2-em-3-carboxylic acid and cyclohexylacetoxymethyl chloride. (Reaction temperature: 60° C., Reaction time: 2 hours, and Yield: 76%)

¹H-NMR (200 MHz, CDCl₃) δ 0.81-1.00 (m, 2H), 1.00-1.23 (m, 3H), 1.16 (d, J=7.2 Hz, 3H), 1.25 (d, J=7.3 Hz, 3H), 1.55-1.81 (m, 5H), 2.18 (d, J=6.9 Hz, 2H), 3.01-3.14 (m, 2H), 3.10 (quint., 1H, J=7.25 Hz), 3.17-3.24 (m, 2H), 3.60 (s, 2H), 3.68-3.81 (m, 2H), 3.82-4.01 (m, 1H), 4.18-4.23 (m, 4H), 4.60-4.72 (m, 1H), 5.83 (d, J=5.61 Hz, 5.61 (d, J=5.61 Hz, 1H), 7.00 (m, 2H), 7.22-7.30 (m, 2H).

### EXAMPLE 5

### Preparation of (1-methylcyclohexanecarboxy)methyl(1R,5S,6S)-2-[(1-(4-fluorobenzyl)azetidin-3-yl)thio]-6-[(R)-1-hydroxyethyl]-1-methyl-carbapen-2-em-3-carboxylate

The title compound was prepared in the same manner as in Example 3, using (1R,5S,6S)-2-[(1-(4-fluorobenzyl)azetidin-3-yl)thio]-6-[(R)-1-hydroxyethyl]-1-methyl-carbapen-2-em-3-carboxylic acid and (1-methylcyclohexanecarboxy)methyl chloride. (Reaction temperature: 65° C., Reaction time: 2 hours, and Yield: 72%)

¹H-NMR (200 MHz, CDCl₃) δ 1.11-1.54 (m, 8H), 1.16 (s, 3H), 1.21 (d, J=7.2 Hz, 3H), 1.31 (d, J=7.3 Hz, 3H), 1.55-1.81 (m, 2H), 2.21 (d, J=6.9 Hz, 2H), 3.01-3.14 (m, 2H), 3.17-3.24 (m, 2H), 3.60 (s, 2H), 3.68-3.81 (m, 2H), 3.82-4.01 (m, 1H), 4.18-4.23 (m, 4H), 4.60-4.72 (m, 1H), 5.84 (d, J=5.61 Hz, 5.60 (d, J=5.61 Hz, 1H), 7.00 (m, 2H), 7.22-7.30 (m, 2H).

### EXAMPLE 6

### Preparation of isovaleroylmethyl(1R,5S,6S)-2-[(1-(4-fluorobenzyl)azetidin-3-yl)thio]-6-[(R)-1-hydroxyethyl]-1-methyl-carbapen-2-em-3-carboxylate

The title compound was prepared in the same manner as in Example 3, using (1R,5S,6S)-2-[(1-(4-fluorobenzyl)azetidin-3-yl)thio]-6-[(R)-1-hydroxyethyl]-1-methyl-carbapen-2-em-3-carboxylic acid and isovaleroylmethyl chloride. (Reaction temperature: to 70° C., Reaction time: 2 hours, and Yield: 76%)

¹H-NMR (200 MHz, CDCl₃) δ 0.94 (d, J=6.6 Hz, 6H), 1.17 (d, J=7.2 Hz, 3H), 1.26 (d, J=7.26 Hz, 3H), 2.05-2.15 (m, 1H), 2.23 (d, J=6.6 Hz, 2H), 3.02-3.0 (m, 1H), 3.18-3.30 (m, 2H), 3.59 (s, 2H), 3.64-3.80 (m, 2H), 3.82-4.01 (m, 2H), 4.12-4.28 (m, 2H), 4.85-4.94 (m, 1H), 5.85 (d, J=5.61 Hz, 5.91 (d, J=5.61 Hz, 1H), 7.05 (m, 2H), 7.22-7.31 (m, 2H).

### EXAMPLE 7

### Preparation of n-decanoyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate

The title compound was prepared in the same manner as in Example 3, using (1R,5S,6S)-2-[(1-(4-fluorobenzyl)azetidin-3-yl)thio]-6-[(R)-1-hydroxyethyl]-1-methyl-carbapen-2-em-3-carboxylic acid and n-decanoyloxymethyl chloride. (Reaction temperature: 65° C., Reaction time: 2 hours, and Yield: 81%)

¹H-NMR (200 MHz, CDCl₃) δ 0.78-0.82 (m, 3H), 1.16 (d, J=7.2 Hz, 3H), 1.17-1.23 (m, 12H), 1.27 (d, J=7.26 Hz, 3H), 1.51-1.58 (m, 2H), 2.31 (t, J=7.58 Hz, 2H), 3.03-3.17 (m, 1H), 3.18-3.30 (m, 2H), 3.59 (s, 2H), 3.64-3.81 (m, 2H), 3.82-4.01 (m, 2H), 4.13-4.29 (m, 2H), 4.85-4.95 (m, 1H), 5.77 (d, J=5.61 Hz, 5.86 (d, J=5.61 Hz, 1H), 7.06 (m, 2H), 7.22-7.33 (m, 2H).

### EXAMPLE 8

### Preparation of 1-(n-hexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate

The title compound was prepared in the same manner as in Example 3, using (1R,5S,6S)-2-[(1-(4-fluorobenzyl)azetidin-3-yl)thio]-6-[(R)-1-hydroxyethyl]-1-methyl-carbapen-2-em-3-carboxylic acid and 1-(n-hexyloxycarbonyloxy)ethyl chloride. (Reaction temperature: 70° C., Reaction time: 2 hours, and Yield: 65%)

¹H-NMR (200 MHz, CDCl₃) δ 0.79-0.81 (m, 3H), 1.15 (d, J=7.2 Hz, 3H), 1.16-1.27 (m, 9H), 1.27 (d, J=7.26 Hz, 3H), 1.51-1.62 (m, 3H), 3.03-3.17 (m, 2H), 3.17-3.30 (m, 2H), 3.59 (s, 2H), 3.64-3.83 (m, 2H), 3.82-4.01 (m, 2H), 4.13-4.29 (m, 2H), 4.85-4.95 (m, 1H), 6.77-6.83 (m, 1H), 7.05 (m, 2H), 7.20-7.33 (m, 2H).

### EXAMPLE 9

### Preparation of 1-(acetoxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate

(1R,5S,6S)-2-[(1-(4-fluorobenzyl)azetidin-3-yl)thio]-6-[(R)-1-hydroxyethyl]-1-methyl-carbapen-2-em-3-carboxylic acid (5 g, 12.3 mmol) was dissolved in N,N-dimethylacetamide (20 mL). Tetrabutylammonium bromide (6 g, 18.45 mmol) was added to the solution, which was then stirred. 1-(Acetoxy)ethyl bromide (2.7 g, 16 mmol) and N,N-diisopropylethylamine (2.6 mL, 18.45 mmol) were added to the reaction mixture, which was then stirred at 35° C. for 2 hours. The reaction mixture was cooled to room temperature and then extracted with water (100 mL) and ethyl acetate (100 mL). The separated organic layer was dried over, anhydrous magnesium sulfate and then concentrated under a reduced pressure. The resulting residue was subjected to silica gel column chromatography (eluent: dichloromethane:acetone=4:1, v/v) for purification to obtain 4.6 g of the title compound (yield: 77%).

¹H-NMR (300 MHz, DMSO-d₆) δ 1.05 (d, 3H), 1.13 (d, 3H), 1.43 (d, 3H), 1.96 (s, 3H), 3.03 (m, 1H), 3.22 (m, 1H), 3.35-3.69 (m, 4H), 3.59 (s, 2H), 3.93 (m, 1H), 4.05 (m, 1H), 4.12 (m, 1H), 5.07 (m, 1H), 6.80 (m, 1H), 7.12 (m, 2H), 7.28 (m, 2H)

### EXAMPLE 10

### Preparation of Phosphoric Acid Salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate

Pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.19 mmol) was dissolved in acetone (1 ml). Phosphoric acid (0.02 ml, 0.2 mmol) was added at 5° C. to the solution, which was then stirred for 30 minutes. Water (1 ml) was added to the reaction mixture. The reaction mixture was distilled under a reduced pressure to remove the organic solvent and then filtered. The obtained solid was washed with water and then dried in a vacuum to obtain 94 mg of the title compound as a white crystal (yield: 80%).

m.p. 124° C.;

¹H NMR (200 MHz, DMSO-d₆) δ 1.25 (t, 11H), 1.38 (d, 3H), 1.89 (br, 1H), 4.3-3.6 (m, 8H), 4.65 (m, 3H), 5.87 (d, 2H), 7.24 (t, 2H), 7.64 (t, 2H)

### EXAMPLE 11

### Preparation of Hydrochloric Acid Salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate

Pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.19 mmol) was dissolved in ethyl acetate (1 ml). 5 ml of hydrochloric acid solution in ethyl acetate was added at 0° C. to the solution, which was then stirred for 30 minutes. The reaction mixture was distilled under a reduced pressure. The resulting residue was dissolved in ethyl acetate (1 ml), and n-hexane (10 ml) was added thereto. The resultant mixture was distilled under a reduced pressure to obtain 0.53 g of the title compound as a powder (yield: 50%).

m.p. 130° C. decomposed;

¹H NMR (200 MHz, DMSO-d₆) δ 1.25 (t, 11H), 1.38 (d, 3H), 1.89 (br, 1H), 4.3-3.6 (m, 8H), 4.65 (m, 3H), 5.87 (d, 2H), 7.24 (t, 2H), 7.64 (t, 2H)

### EXAMPLE 12

### Preparation of Hydrochloric Acid Salt of (isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate

1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.19 mmol) and 5 ml of hydrochloric acid solution in ethyl acetate were reacted in the same manner as in Example 11 to obtain 80 mg of the title compound as a powder (yield: 73%).

m.p. 110° C. decomposed;

¹H NMR (200 MHz, CDCl₃) δ 0.84 (m, 3H), 1.11-1.27 (m, 9H), 1.54-1.57 (m, 3H), 3.82-4.81 (m, 12H), 6.86 (m, 1H), 7.00-7.19 (t, 2H), 7.21-7.26 (m, 2H)

### EXAMPLE 13

### Preparation of Hydrochloric Acid Salt of 1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate

1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.15 mmol) and 5 ml of hydrochloric acid solution in ethyl acetate were reacted in the same manner as in Example 11 to obtain 81 mg of the title compound as a powder (yield: 90%).

m.p. 117° C. decomposed;

¹H NMR (200 MHz, CDCl₃) δ 1.16 (d, 3H), 1.11-1.48 (m, 16H), 3.77-4.88 (m, 12H), 6.83-6.86 (m, 1H), 7.07-7.20 (m, 2H), 7.37-7.44 (m, 2H)

### EXAMPLE 14

### Preparation of Maleic Acid Salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate

Pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.19 mmol) was dissolved in ethyl acetate (1 ml), and then the solution was cooled to 0° C. Maleic acid (23.4 mg, 0.20 mmol) was added to the reaction mixture, which was then stirred for 5 hours. The reaction mixture was distilled under a reduced pressure, and the resulting residue was dissolved in methylene chloride (1 ml). n-Hexane (10 ml) was slowly added to the reaction mixture, which was then stirred at room temperature for 1 hour. The reaction mixture was filtered. The obtained solid was washed with n-hexane and then dried under a reduced pressure to obtain 60 mg of the title compound as a powder (yield: 50%).

m.p. 107-109° C.;

¹H NMR (200 MHz, CD₃OD) δ 1.21-1.31 (m, 15H), 3.23-3.35 (m, 2H), 3.88-3.96 (m, 2H), 4.21-4.48 (m, 4H), 4.48-4.59 (m, 2H), 4.64 (m, 1H), 5.77-5.88 (dd, J=5.69 Hz, 2H), 6.34 (s, 2H), 7.09-7.17 (t, J=8.54 Hz, 2H), 7.29-7.47 (m, 2H)

### EXAMPLE 15

### Preparation of Maleic Acid Salt of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate

1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.19 mmol) was dissolved in isopropyl alcohol (1 ml). Maleic acid (22.7 mg, 0.19 mmol) was added to the reaction mixture, which was then stirred at room temperature for 5 hours. The reaction mixture was distilled under a reduced pressure, and the resulting residue was dissolved in methylenechloride (1 ml). n-Hexane (10 ml) was slowly added to the reaction mixture, which was then filtered. The obtained solid was washed with n-hexane and then dried under a reduced pressure to obtain 110 mg of the title compound as a powder (yield: 93%).

m.p. 129-130° C.,

¹H NMR (200 MHz, CDCl₃) δ 0.84 (m, 3H), 1.11-1.27 (m, 9H), 1.54-1.57 (m, 3H), 3.82-4.81 (m, 12H), 6.34 (s, 2H), 6.86 (m, 1H),7.00-7.19 (t, 2H), 7.21-7.26 (m, 2H)

### EXAMPLE 16

### Preparation of Maleic Acid Salt of 1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate

1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.18 mmol) was dissolved in isopropyl alcohol (1 ml). Maleic acid (21.7 mg, 0.18 mmol) was added to the reaction mixture, which was then stirred at room temperature for 5 hours. Then, 113 mg of the title compound was obtained as a powder, in the same manner as in Example 15 (yield: 93%).

m.p. 134-135° C.;

¹H NMR (200 MHz, CDCl₃) δ 1.16 (d, 3H), 1.11-1.48 (m, 16H), 3.77-4.61 (m, 12H), 6.32 (s, 2H), 6.83-6.86 (m, 1H), 7.08-7.15 (m, 2H), 7.27-7.41 (m, 2H)

### EXAMPLE 17

### Preparation of Fumaric Acid Salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate

Pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.19 mmol) was dissolved in isopropyl alcohol (1 ml). Fumaric acid (23.4 mg, 0.20 mmol) was added to the reaction mixture, which was then stirred at room temperature for 3 hours. n-Hexane (12 ml) was slowly added to the reaction mixture to form a precipitate. The precipitate obtained by filtration was washed with n-hexane and then dried to obtain 60 mg of the title compound as a powder (yield: 50%).

m.p. 110-115° C.;

¹H NMR (200 MHz, CD₃OD) δ 1.25 (m, 12H), 1.39 (d, 3H), 3.65 (m, 2H), 3.82 (m, 1H), 4.13 (m, 3H), 4.18-4.37 (m, 5H), 5.77-5.88 (dd, J=5.69 Hz, 10.17 Hz, 2H), 6.70 (s, 2H), 7.07-7.20 (t, J=8.95 Hz, 2H), 7.38-7.48 (m, 2H)

### EXAMPLE 18

### Preparation of Fumaric Acid Salt of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate

1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.19 mmol) was dissolved in isopropyl alcohol (1 ml). Fumaric acid (22.7 mg, 0.19 mmol) was added to the reaction mixture, which was then stirred at room temperature for 3 hours. The reaction mixture was distilled under a reduced pressure. The resulting residue was dissolved in methylene chloride (1 ml), and n-hexane (10 ml) was added thereto to form a precipitate. The precipitate obtained by filtration was washed with n-hexane and then dried to obtain 115 mg of the title compound as a powder (yield: 93%).

m.p. 162-165° C.;

¹H NMR (200 MHz, CDCl₃) δ 0.84 (m, 3H), 1.11-1.27 (m, 9H), 1.54-1.57 (m, 3H), 3.82-4.81 (m, 12H), 6.75 (s, 2H), 6.86 (m, 1H), 7.00-7.19 (t, 2H), 7.21-7.26 (m, 2H)

### EXAMPLE 19

### Preparation of Fumaric Acid Salt of 1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate

1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.18 mmol) was dissolved in isopropyl alcohol (1 ml). Fumaric acid (21.7 mg, 0.18 mmol) was added to the reaction mixture, which was then stirred at room temperature for 3 hours. Then, 115 mg of the title compound was obtained as a powder, in the same manner as in Example 18 (yield: 95%).

m.p. 157-158° C.;

¹H NMR (200 MHz, CDCl₃) δ 1.16 (d, 3H), 1.11-1.88 (m, 16H), 3.77-4.62 (m, 12H), 6.79 (s, 2H), 6.83-6.86 (m, 1H), 7.07-7.15 (m, 2H), 7.27-7.41 (m, 2H)

### EXAMPLE 20

### Preparation of Benzenesulfonic Acid Salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate

Pivaloyloxymethyl(1R,5S,6S)-2-[(1-(4-fluorobenzyl)azetidin-3-yl_thio]-6-[(R)-1-hydroxyethyl]-1-methyl-carbapen-2-em-3-carboxylate (100 mg, 0.19 mmol) was dissolved in isopropyl alcohol (1 ml). Benzenesulfonic acid (31.9 mg, 0.20 mmol) was added to the reaction mixture, which was then stirred at room temperature for 5 hours. n-Hexane (12 ml) was added to the reaction mixture to form a precipitate. The precipitate obtained by filtration was washed with n-hexane and then dried to obtain 99.3 mg of the title compound as a powder (yield: 77%).

m.p. 113-115° C.;

¹H NMR (200 MHz, CD₃OD) δ 1.25 (m, 11H), 1.39 (d, 3H), 3.68-4.28 (m, 11H), 5.77-5.89 (dd, J=5.69 Hz, 10.17 Hz, 2H), 6.97-7.05 (m, 5H), 7.23-7.31 (m, 4H)

### EXAMPLE 21

### Preparation of Benzenesulfonic Acid Salt of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate

1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.19 mmol) was dissolved in isopropyl alcohol (1 ml). Benzenesulfonic acid (30.9 mg, 0.19 mmol) was added to the reaction mixture, which was then was stirred at room temperature for 5 hours. Then, 109.5 mg of the title compound was obtained as a powder, in the same manner as in Example 20 (yield: 83).

m.p. 105-106° C.;

¹H NMR (200 MHz, CDCl₃) δ 0.84 (m, 3H), 1.11-1.27 (m, 9H), 1.54-1.57 (m, 3H), 3.82-4.81 (m, 12H), 6.86-6.97 (m, 4H), 7.26-7.39 (m, 4H), 7.79-7.83 (m, 2H)

### EXAMPLE 22

### Preparation of Benzenesulfonic Acid Salt of 1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate

1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.18 mmol) was dissolved in isopropyl alcohol (1 ml). Benzenesulfonic acid (29.6 mg, 0.18 mmol) was added to the reaction mixture, which was then stirred at room temperature for 5 hours. Then, 110 mg of the title compound was obtained as a powder, in the same manner as in Example 20 (yield: 85%).

m.p. 120-121° C.;

¹H NMR (200 MHz, CDCl₃) δ 1.16 (d, 3H), 1.11-1.88 (m, 16H), 3.77-4.72 (m, 12H), 6.86-6.88 (m, 1H), 7.04-7.10 (m, 3H), 7.38-7.45 (m, 4H), 7.71-7.75 (d, 2H)

### EXAMPLE 23

### Preparation of p-toluenesulfonic Acid Salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate

Pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.19 mmol) was dissolved in isopropyl alcohol (2 ml). p-Toluenesulfonic acid (34.7 mg, 0.20 mmol) was added to the reaction mixture, which was then stirred at room temperature for 5 hours. n-Hexane (12 ml) was slowly added to the reaction mixture to form a precipitate. The precipitate obtained by filtration was washed with n-hexane and then dried to obtain 109 mg of the title compound as a powder (yield: 83%).

m.p. 120-121° C.;

¹H NMR (200 MHz, CD₃OD) δ 1.25 (m, 11H), 1.39 (d, 3H), 2.34 (s, 3H), 3.85-4.65 (m, 11H), 5.77-5.88 (dd, J=5.69 Hz, 10.17 Hz, 2H), 7.10-7.22 (m, 4H), 7.62-7.72 (m, 4H)

### EXAMPLE 24

### Preparation of p-toluenesulfonic Acid Salt of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate

1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.19 mmol) was dissolved in isopropyl alcohol (1 ml). p-Toluenesulfonic acid (33.6 mg, 0.19 mmol) was added to the reaction mixture, which was then stirred at room temperature for 5 hours. Then, 121 mg of the title compound was obtained as a powder, in the same manner as in Example 23 (yield: 90%).

m.p. 129-130° C.;

¹H NMR (200 MHz, CDCl₃) δ 1.16 (d, 3H), 1.11-1.88 (m, 16H), 2.37 (s, 3H), 3.77-4.72 (m, 12H), 6.86-6.88 (m, 1H), 7.00-7.09 (t, 2H), 7.17-7.21 (d, 2H), 7.38-7.45 (d, 2H)

### EXAMPLE 25

### Preparation of p-toluenesulfonic Acid Salt of 1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate

1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.18 mmol) was dissolved in isopropyl alcohol (1 ml). p-Toluenesulfonic acid (32.2 mg, 0.18 mmol) was added to the reaction mixture, which was then stirred at room temperature for 5 hours. Then, 112 mg of the title compound was obtained as a powder, in the same manner as in Example 23 (yield: 85%).

m.p. 129-130° C.;

¹H NMR (200 MHz, CDCl₃) δ 1.16 (d, 3H), 1.11-1.88 (m, 16H), 2.37 (s, 3H), 3.77-4.72 (m, 12H), 6.86-6.88 (m, 1H), 7.00-7.09 (t, 2H), 7.17-7.21 (d, 2H), 7.38-7.45 (d, 2H)

### EXAMPLE 26

### Preparation of Trifluoroacetic Acid Salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate

Pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.19 mmol) was dissolved in ethyl acetate (1 ml) and the resulting solution was cooled to 0° C. Trifluoroacetic acid (26.3 mg, 0.23 mmol) was added to the reaction mixture, which was then stirred at room temperature for 50 minutes. The reaction mixture was cooled to 0° C. and then n-hexane (12 ml) was slowly added thereto to form a precipitate. The reaction mixture was stirred at the same temperature for 1 hour and then filtered. The obtained precipitate was washed with n-hexane and then dried to obtain 112 mg of the title compound as a powder (yield: 93%).

m.p. 80-81° C.;

¹H NMR (200 MHz, CDCl₃) δ 1.25 (m, 12H), 1.39 (d, 3H), 3.65-4.45 (m, 11H), 5.80-5.91 (dd, J=5.69 Hz, 10.17 Hz, 2H), 7.07-7.16 (t, J=8.95 Hz, 2H), 7.40-7.44 (m, 2H)

### EXAMPLE 27

### Preparation of Trifluoroacetic Acid Salt of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate

1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.19 mmol) was dissolved in ethyl acetate (1 ml) and the resulting solution was cooled to 0° C. Trifluoroacetic acid (25.5 mg, 0.22 mmol) was added to the reaction mixture, which was then stirred at room temperature for 50 minutes. Then, 114 mg of powder of the title compound was obtained as a powder, in the same manner as in Example 26 (yield: 93%).

m.p. 70-71° C.;

¹H NMR (200 MHz, CDCl₃) δ 0.84 (m, 3H), 1.11-1.27 (m, 9H), 1.42-1.58 (m, 3H), 3.95-4.88 (m, 12H), 6.86 (m, 1H), 7.01-7.18 (m, 2H), 7.28-7.38 (m, 2H)

### EXAMPLE 28

### Preparation of Trifluoroacetic Acid Salt of 1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate

1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.19 mmol) was dissolved in ethyl acetate (1 ml) and the resulting solution was cooled to 0° C. Trifluoroacetic acid (25.5 mg, 0.22 mmol) was added to the reaction mixture, which was then stirred at room temperature for 50 minutes. Then, 115 mg of the title compound was obtained as a powder, in the same manner as in Example 26 (yield: 95%).

m.p. 85-86° C.;

¹H NMR (200 MHz, CDCl₃) δ 1.16 (d, 3H), 1.11-1.88 (m, 16H), 3.80-4.83 (m, 12H), 6.83-6.86 (m, 1H), 7.07-7.17 (t, 2H), 7.37-7.44 (d, 2H)

### EXAMPLE 29

### Preparation of Lactic Acid Salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate

Pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.19 mmol) was dissolved in isopropyl alcohol (2 ml). Lactic acid (18.2 mg, 0.20 mmol) was added to the reaction mixture, which was then stirred at room temperature for 5 hours. n-Hexane (12 ml) was slowly added to the reaction mixture to form a precipitate. The precipitate obtained by filtration was washed with n-hexane and then dried to obtain 79 mg of the title compound as a powder (yield: 68%).

m.p. 111-112° C.;

¹H NMR (200 MHz, CDCl₃) δ 1.25 (m, 12H), 1.39 (m, 6H), 3.65-4.40 (m, 12H), 5.77-5.88 (dd, J=5.69 Hz, 10.17 Hz, 2H), 07-7.20 (t, J=8.95 Hz, 2H), 7.38-7.48 (m, 2H)

### EXAMPLE 30

### Preparation of Lactic Acid Salt of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate

Isopropyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.19 mmol) was dissolved in isopropyl alcohol (1 ml). Lactic acid (20.0 mg, 0.22 mmol) was added to the reaction mixture, which was then stirred at room temperature for 5 hours. Then, 82 mg of the title compound was obtained as a powder, in the same manner as in Example 29 (yield: 69%).

m.p. 110-112° C.;

¹H NMR (200 MHz, CDCl₃) δ 0.84 (m, 3H), 1.11-1.27 (m, 12H), 1.42-1.58 (m, 3H), 3.95-4.88 (m, 13H), 6.86 (m, 1H), 7.01-7.18 (m, 2H), 7.28-7.38 (m, 2H)

### EXAMPLE 31

### Preparation of Lactic Acid Salt of 1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate

1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate (100 mg, 0.19 mmol) and lactic acid (16.8 mg, 0.19 mmol) were reacted in the same manner as in Example 29 to obtain 80 mg of the title compound as a powder (yield: 63%).

m.p. 115-117° C.;

¹H NMR (200 MHz, CDCl₃) δ 1.16 (d, 3H), 1.11-1.88 (m, 19H), 3.81-4.80 (m, 13H), 6.83-6.85 (m, 1H), 7.08-7.17 (t, 2H), 7.38-7.45 (d, 2H)

### TEST EXAMPLE 1

### Test of Pharmacokinetics

The pharmacokinetics of the compounds of the present invention was determined using mice. Each of the compounds of Examples 1 to 3 was dissolved in 25% ethanol, and mice were orally administered (PO) or subcutaneously injected (SC) at a dosage of 40 mg/kg body weight (I.C.R mice, weighing 22 to 25 g, 3 mice/group). Blood samples were collected from mice tails at 10 min., 20 min., 30 min., 45 min., 1 hour, 1.5 hours, 2 hours, 3 hours and 4 hours after the administration. As a comparative example, (1R,5S,6S)-2-[(1-(4-fluorobenzyl)azetidin-3-yl)thio]-6-[(R)-1-hydroxyethyl]-1-methyl-carbapen-2-em-3-carboxylic acid prepared in Preparation Example 4 was dissolved in distilled water, and mice were orally administered (PO) or subcutaneously injected (SC) at a dose of 40 mg/kg body weight (I.C.R mice, weighing 22 to 25 g, 4 mice/group). Blood samples were collected from mice tails in the same manner described above.

The concentrations of each compound in blood were measured using bioassay methods: Agar plate was prepared with agar medium containing 1% Streptococcus Pyogenes 77A culture solution, and the blood samples and the diluted standards (each of which were obtained by dilution of the already-known concentration by two times) were added to wells formed on the plates. The plate was stored at 4° C. for 1 hour to is allow the sample to spread, and incubated at 37° C. for 18 hours. Diameters of each inhibition-circle were measured and then the concentration of the compounds in blood was calculated, based on the calibration curve obtained from the diluted standards. The obtained pharmacokinetic parameters (Cmax, Tmax, T_{1/2}, and AUC) are shown in Table 1 below.

TABLE 1

| | | AUC*¹ | Tₘₐₓ*² | Cₘₐₓ*³ | T_{1/2}*⁴ |
|---|---|---|---|---|---|
| Compounds | Administration | (μg · h/ml) | (hr) | (μg/ml) | (hr) |
| Preparation | SC | 83.24 | 0.38 | 68.5 | 0.76 |
| Example 4 | PO | 8.28 | 0.56 | 3.97 | 1.32 |
| Example 1 | SC | 226 | 1.76 | 97.9 | 6.28 |
| | PO | 117 | 0.38 | 88.1 | 0.931 |
| Example 2 | SC | 174 | 0.75 | 66.4 | 3.92 |
| | PO | 94.8 | 1.00 | 72.8 | 1.15 |
| Example 3 | SC | 56.9 | 2.50 | 16.2 | 1.05 |
| | PO | 64.5 | 0.59 | 39.0 | 1.43 |

*¹area under blood concentration curve
*²time at the point of maximum blood concentration
*³maximum blood concentration
*⁴half time of blood concentration

As a result of HPLC assay of blood samples, the test compounds were found to exist in the metabolite form (i.e., (1R,5S,6S)-2-[(1-(4-fluorobenzyl)azetidin-3-yl)thio]-6-[(R)-1-hydroxyethyl]-1-methyl-carbapen-2-em-3-carboxylic acid) in the blood samples. As shown in Table 1, the ratios of oral/subcutaneous AUCs of each compound of Examples 1 to 3 were about 52%, about 54%, and about 113%, respectively. Thus, the compounds of the present invention have excellent oral absorption

### TEST EXAMPLE 2

### Measurement of Minimum Inhibitory Concentration (MIC)

Referring to the results of Test Example 1, when the 2-arylmethylazetidine-carbapenem-3-carboxylic acid derivative according to the present invention is orally administered, it is absorbed through the gastrointestinal tract in high absorption rate and then metabolized into 2-arylmethylazetidine-carbapenem-3-carboxylic acid, that is, (1R,5S,6S)-2-[(1-(4-fluorobenzyl)azetidin-3-yl)thio]-6-[(R)-1-hydroxyethyl]-1-methyl-carbapen-2-em-3-carboxylic acid, in vivo.

The metabolite has a wide spectrum of antibacterial activities against Gram-negative and Gram-positive bacteria and excellent antibacterial activities against resistant bacteria such as methicillin-resistant Staphylococcus aurus (MRSA) and quinolone-resistant strains (QRS), as in WO2006/025634 by the present inventors. The minimum inhibitory concentrations of the metabolite, i.e., (1R,5S,6S)-2-[(1-(4-fluorobenzyl)azetidin-3-yl)thio]-6-[(R)-1-hydroxyethyl]-1-methyl-carbapen-2-em-3-carboxylic acid, against various strains (mainly clinical isolated strains), were also measured in the same manner as in WO2006/025634, and the results are shown in Table 2.

TABLE 2
Minimum Inhibitory Concentration (MIC)

| Strains (number of strains) | MIC (g/ml) |
|---|---|
| Staphylococcus aureus (2) | 0.002 |
| Staphylococcus aureus ATCC 29213 | 0.015 |
| Staphylococcus aureus (10), MRSA | 0.025-3.125 |
| Staphylococcus aureus (5), QRS | 0.049-0.391 |
| Streptococcus pyogenes (2)) | 0.013-0.025 |
| Streptococcus faecium (1) | 1.563 |
| Streptococcus pneumoniae Pen. R (4) | 0.12 |
| Streptococcus pneumoniae Pen. S (4) | <0.008 |
| Streptococcus Levo. R (4) | 0.006-0.12 |
| Streptococcus Levo. S (4) | <0.008-0.25 |
| Streptococcus ATCC 496 (4) | 0.03 |
| MRCNS (2) | 0.5-8 |
| GBBS ATCC 12386 | 0.03 |
| Escherichia coli (4) | 0.025-0.098 |
| Salmonella typhimurium 179 | 0.049 |
| Klebsiella oxytoca 1082 | 0.049 |
| Klebsiella pneumoniae (4) | 0.06-0.25 |
| Klebsiella pneumoniae ATCC 13883 | 0.1225 |
| Enterobacter cloacae P 99(2)3 | 0.049 |
| Enterobacter cloacae ATCC 13880 | 2 |
| Moganella morganii (2) | 0.25-1 |
| Moraxella catarrhalis AMP R(3) | 0.03-0.12 |
| Moraxella catarrhalis AMP S(3) | <0.008 |
| Psudomonas aeruginosa (4) | 32-64 |

As can be seen in Table 2, the metabolite of compounds according to the present invention has a broad spectrum of antibacterial activities against Gram-positive and Gram-negative bacteria and has excellent antibacterial activities against MRSA and QRS, particularly staphylococcus, Streptococcus, and Klebsiella strains.

### TEST EXAMPLE 3

### Powder X-Ray Diffractometry

As a result of observing the phosphoric acid salt of pivaloyloxymethyl(1R,5S,6S)-2-[(1-(4-fluorobenzyl)azetidin-3-yl)thio]-6-[(R)-1-hydroxyethy]-1-methyl-carbapen-2-em-3-carboxylate prepared in Example 10 using a polar microscope, crystalline form thereof was identified. The powder X-ray diffraction results are shown in Table 3 below.

TABLE 3

| | d-spacing | | Intensity | |
|---|---|---|---|---|
| 2θ | (Å) | Intensity value | (I/I₀) | FWHM |
| 7.0600 | 12.5110 | 2027 | 338 | 0.2600 |
| 8.6400 | 10.2260 | 5363 | 1000 | 0.2200 |
| 9.8000 | 9.0180 | 1453 | 192 | 0.1200 |
| 10.1200 | 8.7337 | 950 | 90 | 0.3000 |
| 10.9600 | 8.0659 | 993 | 105 | 0.2600 |
| 12.6400 | 6.9975 | 1024 | 95 | 0.2600 |
| 14.1000 | 6.2759 | 1509 | 158 | 0.0800 |
| 15.1600 | 5.8395 | 1340 | 85 | 0.2800 |
| 15.7000 | 5.6399 | 3521 | 519 | 0.3400 |
| 16.5600 | 5.3488 | 2849 | 377 | 0.2800 |
| 17.6000 | 5.0350 | 1857 | 161 | 0.2800 |
| 18.2200 | 4.8651 | 2273 | 272 | 0.2800 |
| 19.2600 | 4.0047 | 1965 | 212 | 0.2400 |
| 19.8200 | 4.4757 | 4396 | 768 | 0.1000 |
| 20.1600 | 4.3968 | 2727 | 370 | 0.1200 |
| 20.4800 | 4.3330 | 1733 | 170 | 0.2000 |
| 22.9200 | 3.8769 | 1663 | 149 | 0.2000 |
| 23.3200 | 3.8113 | 1352 | 90 | 0.2200 |
| 24.4400 | 3.6392 | 1491 | 138 | 0.2200 |
| 24.6600 | 3.5786 | 1247 | 97 | 0.2000 |
| 25.8800 | 3.4399 | 2279 | 2279 | 0.2800 |
| 26.3500 | 3.3783 | 1269 | 1269 | 0.1400 |
| 28.8500 | 3.0911 | 1303 | 1303 | 0.3800 |
| 30.0500 | 2.9704 | 1086 | 1086 | 0.2800 |
| 30.6600 | 2.9136 | 1027 | 1027 | 0.2400 |

### TEST EXAMPLE 4

### Chemical Stability Test

Stability test of the phosphoric acid salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate prepared in Example 10 was performed. 500 mg of the compound prepared in Example 10 was charged into a glass bottle, and HPLC purities of the compound were measured at the condition of 40° C. and a humidity of 75%, at 1 week, 2 week, 3 week, and 1 month. The HPLC purities were obtained by comparing each of the measured HPLC amounts with the initial HPLC amount. The results are shown in Table 4 below.

TABLE 4

| Test | HPLC Purity (%) | | | | |
|---|---|---|---|---|---|
| Compound | Initial | 1 week | 2 week | 3 week | 1 month |
| Example 10 | 99.83 | 99.85 | 99.78 | 99.74 | 99.69 |

As can be seen in Table 4, when the ester derivative is converted into an acid addition salt form, it is found that the acid addition salt shows high chemical stability.

### TEST EXAMPLE 5

### Acute Toxicity Test

The acute toxicity of the compound of Example 1 was tested using several groups of I.C.R. mice each of 10 mice. 500 mg/kg, 1,000 mg/kg, and 2,000 mg/kg doses of the compound of Example 1 were orally administered. The body temperature change, weight change, and death were observed for 7 days after the oral administration. As a result, no mice died, and no distinct body temperature change nor weight loss were observed. Thus, LD₅₀ was placed at a level higher than 2,000 mg/kg. The compound of Example 1 is thus a largely non-toxic antibiotic.

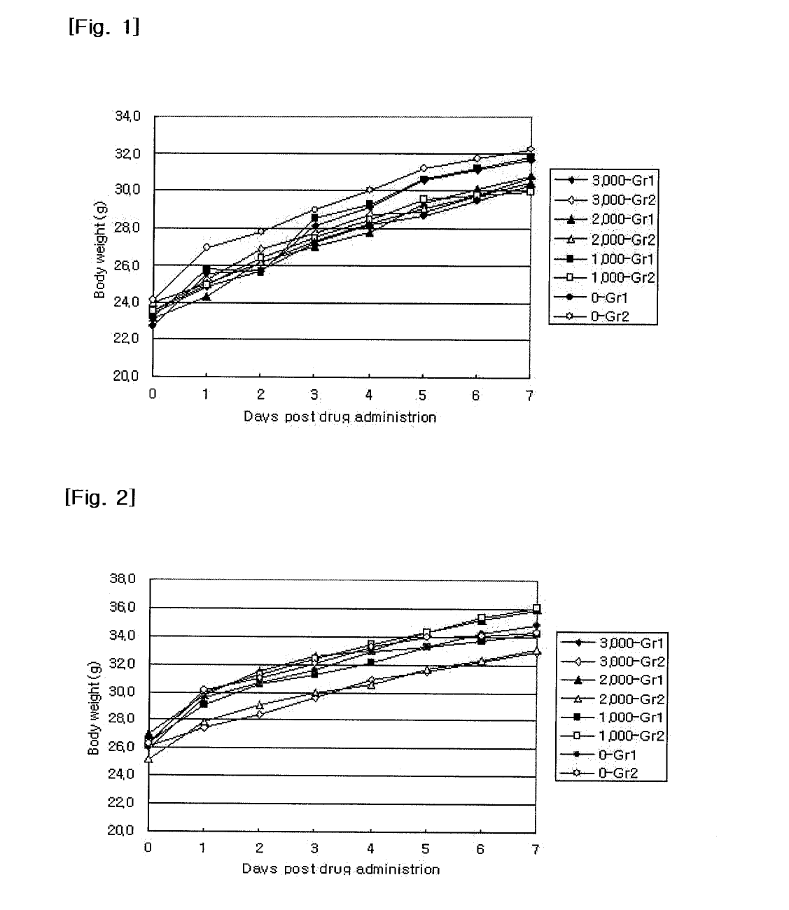

In addition, acute toxicity of the compounds of Examples 10 and 17 was tested using two groups of I.C.R. mice each of 5 mice/2 groups. 1,000 mg/kg, 2,000 mg/kg, and 3,000 mg/kg doses of the compounds of Examples 10 to 17 were orally administered. The body temperature change, weigh change, and death were observed for 7 days after the oral administration. As a result, no mice died, and no body distinct temperature change nor weight loss were observed (FIGS. 1 and 2). Thus, LD₅₀ was placed at a level higher than 3,000 mg/kg. Therefore, the compounds of Examples 10 to and 17 are non-toxic antibiotics.

1. A carbapenem derivative of Formula 1 or its pharmaceutically acceptable salt: 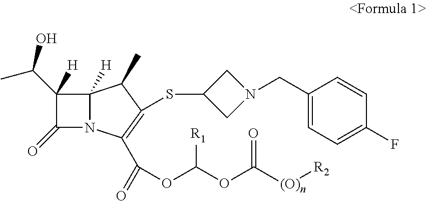
wherein, R₁ is a hydrogen atom or a C₁-C₄ alkyl group; R₂ is a linear or branched C₁-C₁₂ alkyl group optionally substituted with C₄-C₇ cycloalkyl, or a C₄-C₇ cycloalkyl group optionally substituted with C₁-C₄ alkyl; and n is 0 or 1.

2. The carbapenem derivative or its pharmaceutically acceptable salt of claim 1, wherein the pharmaceutically acceptable salt is an acid addition salt of the carbapenem derivative of Formula 1.

3. The carbapenem derivative or its pharmaceutically acceptable salt of claim 2, wherein the acid addition salt is an addition salt of the inorganic acid selected from the group consisting of hydrochloric acid, phosphoric acid, sulfuric acid, hydrobromic acid, hydroiodic acid, and nitric acid; or an addition salt of the organic acid selected from the group consisting of acetic acid, propionic acid, butyric acid, trifluoroacetic acid, trichloroacetic acid, fumaric acid, maleic acid, lactic acid, methanesulfonic acid, trifluoromethanesulfonic acid, benzoic acid, p-nitrobenzoic acid, benzenesulfonic acid, p-nitrobenzenesulfonic acid, p-bromobenzenesulfonic acid, toluenesulfonic acid, 2,4,6-triisopropylbenzenesulfonic acid, and diphenylphosphinic acid.

4. The carbapenem derivative or its pharmaceutically acceptable salt of claim 2, wherein the acid addition salt is an addition salt of phosphoric acid, hydrochloric acid, maleic acid, fumaric acid, benzenesulfonic acid, p-toluenesulfonic acid, trifluoroacetic acid, or lactic acid.

5. The carbapenem derivative or its pharmaceutically acceptable salt of claim 1, which is selected from the group consisting of:
pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;
1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;
1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;
cyclohexylacetoxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;
(1-methylcyclohexanecarboxy)methyl(1R,5S,6S)-2-[(1-(4-fluorobenzyl)azetidin-3-yl)thio]-6-[(R)-1-hydroxyethyl]-1-methyl-carbapen-2-em-3-carboxylate;
isovaleroylmethyl(1R,5S,6S)-2-[(1-(4-fluorobenzyl)azetidin-3-yl)thio]-6-[(R)-1-hydroxyethyl]-1-methyl-carbapen-2-em-3-carboxylate;
n-decanoyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;
1-(n-hexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;
1-(acetoxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;
phosphoric acid salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;
hydrochloric acid salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;
hydrochloric acid salt of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;
hydrochloric acid salt of 1-(cyclohexyloxycarbonyloxy)ethyl (1R,5S,6S)-2-[(1-(4-fluorobenzyl)azetidin-3-yl)thio]-6-[(R)-1-hydroxyethyl]-1-methyl-carbapen-2-em-3-carboxylate;
maleic acid salt of pivaloyloxymethyl (1R,5S,6S)-2-[(1-(4-fluorobenzyl)azetidin-3-yl)thio]-6-[(R)-1-hydroxyethyl]-1-methyl-carbapen-2-em-3-carboxylate;
maleic acid salt of 1-(isopropyloxycarbonyloxy)ethyl (1R,5S,6S)-2-[(1-(4-fluorobenzyl)azetidin-3-yl)thio]-6-[(R)-1-hydroxyethyl]-1-methyl-carbapen-2-em-3-carboxylate;
maleic acid salt of 1-(cyclohexyloxycarbonyloxy)ethyl (1R,5S,6S)-2-[(1-(4-fluorobenzyl)azetidin-3-yl)thio]-6-[(R)-1-hydroxyethyl]-1-methyl-carbapen-2-em-3-carboxylate;
fumaric acid salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;
fumaric acid salt of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;
fumaric acid salt of 1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;
benzenesulfonic acid salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;
benzenesulfonic acid salt of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;
benzenesulfonic acid salt of 1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;
p-toluenesulfonic acid salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;
p-toluenesulfonic acid salt of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;
p-toluenesulfonic acid salt of 1-(cyclohexyloxycarbonyloxy)ethyl(1 R,5S,6S)-2-[(1-(4-fluorobenzyl)azetidin-3-yl)thio]-6-[(R)-1-hydroxyethyl]-1-methyl-carbapen-2-em-3-carboxylate;
trifluoroacetic acid salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;
trifluoroacetic acid salt of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;
trifluoroacetic acid salt of 1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate;
lactic acid salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate;
lactic acid salt of 1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate; and
lactic acid salt of 1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate.

6. The carbapenem derivative or its pharmaceutically acceptable salt of claim 1, which is selected from the group consisting of:
pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate or its acid addition salt;
1-(isopropyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate or its acid addition salt; and
1-(cyclohexyloxycarbonyloxy)ethyl(1R,5S,6S)-2-[(1-(4-fluorobenzyl)azetidin-3-yl)thio]-6-[(R)-1-hydroxyethyl]-1-methyl-carbapen-2-em-3-carboxylate or its acid addition salt.

7. A phosphoric acid salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate.

8. A process for preparing a carbapenem derivative of Formula 1 or its pharmaceutically acceptable salt, which comprises reacting a compound of Formula 2 with a compound of Formula 3: 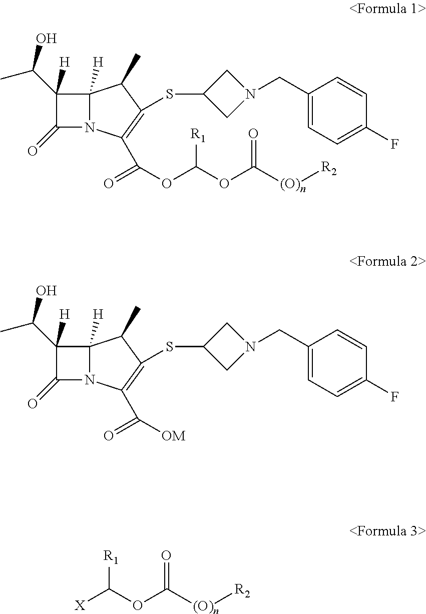
wherein, M is a hydrogen atom or an alkali metal; X is a halogen atom; and R₁, R₂, and n is the same as defined in claim 1.

9. The process of claim 8, wherein the reaction between the compounds of Formulae 2 and 3 is performed in the presence of at least one base selected from the group consisting of sodium hydroxide, potassium hydroxide, sodium carbonate, potassium carbonate, sodium bicarbonate, potassium bicarbonate, triethylamine, N,N-diisopropylethylamine, and pyridine.

10. The process of claim 9, wherein the reaction between the compounds of Formulae 2 and 3 is performed in the presence of at least one quaternary ammonium salt selected from the group consisting of tetraethylammonium chloride, tetrabutylammonium chloride, tetrabutylammonium bromide, and benzyltriethylammonium chloride.

11. The process of claim 8, wherein the reaction between the compounds of Formulae 2 and 3 is performed in the presence of at least one organic solvent selected from the group consisting of diethyl ether, tetrahydrofuran, dioxane, toluene, xylene, cyclohexane, dichloromethane, chloroform, N,N-dimethylformamide, N,N-dimethylacetamide, acetonitrile, and dimethylsulfoxide.

12. A process for preparing an acid addition salt of a carbapenem derivative of Formula 1, which comprises reacting a carbapenem derivative of Formula 1 with an acid: 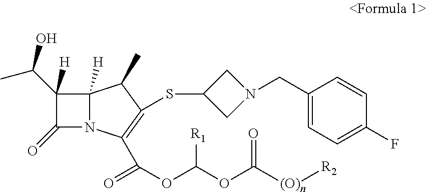
wherein, R₁, R₂, and n is the same as defined in claim 1.

13. The process of claim 12, wherein the acid is an inorganic acid selected from the group consisting of hydrochloric acid, phosphoric acid, sulfuric acid, hydrobromic acid, hydroiodic acid, and nitric acid; or an organic acid selected from the group consisting of acetic acid, propionic acid, butyric acid, trifluoroacetic acid, trichloroacetic acid, fumaric acid, maleic acid, lactic acid, methanesulfonic acid, trifluoromethanesulfonic acid, benzoic acid, p-nitrobenzoic acid, benzenesulfonic acid, p-nitrobenzenesulfonic acid, p-bromobenzenesulfonic acid, toluenesulfonic acid, 2,4,6-triisopropylbenzenesulfonic acid, and diphenylphosphinic acid.

14. The process of claim 12, wherein the acid is phosphoric acid, hydrochloric acid, maleic acid, fumaric acid, benzenesulfonic acid, p-toluenesulfonic acid, trifluoroacetic acid, or lactic acid.

15. The process of claim 12, wherein the reaction is performed in at least one organic solvent selected from the group consisting of acetone, ethyl acetate, isopropyl alcohol, tetrahydrofuran, and acetonitrile.

16. The process according to claim 12, wherein the carbapenem derivative of Formula 1 is prepared according to the process comprising: reacting a compound of Formula 2 with a compound of Formula 3: 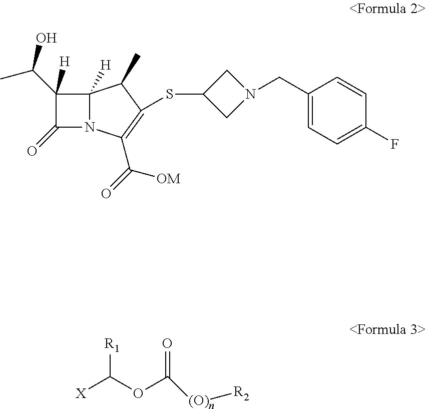
wherein, M is a hydrogen atom or an alkali metal; X is a halogen atom and R₁, R₂ is the same as defined in claim 12, and n is 0 or 1.

17. An antibiotic composition comprising an effective amount of the carbapenem derivative of Formula 1 or its pharmaceutically acceptable salt as defined in claim 1, as an active ingredient; and a pharmaceutically acceptable carrier.

18. The antibiotic composition of claim 17, wherein the pharmaceutically acceptable salt is an acid addition salt of the carbapenem derivative of Formula 1.

19. An antibiotic composition comprising an effective amount of a phosphoric acid salt of pivaloyloxymethyl(1R,5S,6S)-2-[(1-(4-fluorobenzyl)azetidin-3-yl)thio]-6-[(R)-1-hydroxyethyl]-1-methyl-carbapen-2-em-3-carboxylate as an active ingredient and a pharmaceutically acceptable carrier.
